(19) United States
(12) Patent Application Publication
(10) Pub. No.: US 2015/0010957 A1
(43) Pub. Date: Jan. 8, 2015

(54) Alkcell Process (alkali-cellulase process) for the conversion of biomass (cellulosic material) to glucose

(71) Applicant: John Joseph Savarese, Collegeville, PA (US)

(72) Inventor: John Joseph Savarese, Collegeville, PA (US)

(21) Appl. No.: 13/935,743

(22) Filed: Jul. 5, 2013

(51) Int. Cl. C12P 19/14; C12P 7/14

(52) U.S. Cl. CPC C12P 19/14; C12P 7/14
USPC 435/99; 435/162

(57) ABSTRACT

The Alkcell (alkali-cellulase) Process is a method for the conversion of biomass (cellulosic material) such as agricultural waste, for example, corn stover into glucose. The process uses an alkali pretreatment of biomass followed by hydrolysis with cellulase enzymes to produce glucose. The Alkcell Process uses commonly available materials and methods readily implemented near the biomass growing site. The operating conditions are mild with temperatures not above boiling water solutions, and require half-day conversion to produce glucose in water. Remaining solids can be recycled for additional glucose. An advantage of the Alkcell Process is implementation at or near the biomass growing site that avoids transporting biomass distances to special cellulose processing facilities. The glucose produced can be fermented at existing grain fermenting facilities so that special cellulose handling refineries are not required. The Alkcell Process can be configured to a manual or powered process done in individual steps or continuously.

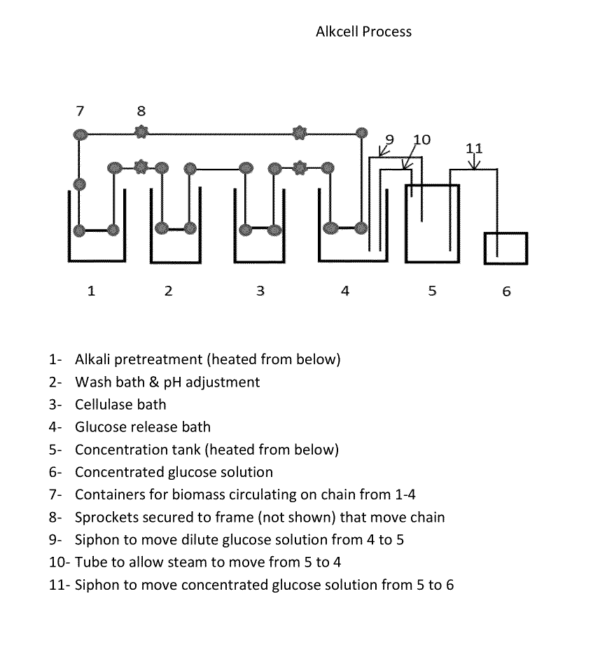

### FEDERALLY SPONSORED RESEARCH OR DEVELOPMENT

No work regarding this work, specification, or application has received federal funding.

### BACKGROUND

According to experts, “For cellulosic ethanol to become a reality, biotechnological solutions should focus on optimizing the conversion of biomass to sugars” [1]. The need to process biomass rather than the cost of the biomass itself is a major economic factor in converting cellulosic materials to fuels such as ethanol [2]. While combining processing steps such as in simultaneous saccharificaton and fermentation can improve economics, albeit with substantial technical difficulties, continuous processes for converting biomass to glucose and further to ethanol are perhaps a better cost reducers; however, little work has been done [3]. One continuous biomass fractionation process for producing ethanol as well as other products has been demonstrated in the laboratory and pilot plant work in ongoing [4]. However, the technology requires sophisticated equipment and extreme operating conditions that can only be managed in a large dedicated bioreactor facility predictably at a distance from growing fields.

Another roadblock to efficient and practical conversion of biomass to ethanol is the cost of transporting the cellulosic material from farm gate to refinery gate. It is estimated that a refinery producing 50 million gallons of ethanol annually would need 135 truck deliveries per day of baled biomass [5]. The social and environmental impact in the area surrounding the facility and along the transport route due to engine emissions alone would be substantial. Transportation cost is estimated at nearly 40% of the delivered feedstock cost [6]. Upwards of $100 is estimated for one truck transporting biomass a distance of 60 km from farm to refinery [7]. An approach to reducing transportation cost is preprocessing at regional centers but is limited to mechanical preparation of the biomass such as cleaning, particle size reduction, and packaging [8]. Even so, the possibility of developing feedstock cooperatives among farmers could provide economies in use of equipment and offer employment during seasons when biomass is not grown.

An approach to solving the transportation problem and the need to have an efficient conversion of biomass to glucose is alkali-cellulose (Alkcell) processing. Recent work has shown the practicality of Alkcell processing locally, that is, at the growing site or at close regional centers to convert biomass to glucose [9]. After concentration with thermal or membrane methods the glucose solution can be transported to existing grain fermenting facilities and used directly without further processing.

The Alkcell process was studied at laboratory scale using batch type operation. However, for routine operation a continuous operation would be most efficient and cost effective to produce a glucose solution with residual solids that can then be recycled to yield more glucose. In a continuous process, the biomass is carried in containers that move through the stages of the process and then back to the start for recycling of residual solids and loading with fresh biomass.

The initial stage of Alkcell processing is immersion of the biomass carrier in an alkali solution for sufficient time and at sufficient temperature to effect adequate solution of lignin and change in biomass structure to allow enzyme hydrolysis. After washing and pH adjustment of the biomass, the carriers are immersed in a cellulase bath. Minimal cellulase loading to provide sufficient binding to cellulose substrate has received some attention [10]. An optimum temperature for adsorption has been explored with conflicting results [11]. To minimize production of glucose in the enzyme bath, small bath volumes, optimum temperature as well as adequate but minimal amounts of cellulase are required for efficient operation. These are critical conditions since the cost of enzyme is a major economic factor [12].

It has been reported that the amount of biomass is not a factor in continuous fermentation of cellulosic biomass to ethanol [13]; however, determination of an optimum biomass load would seem critical for an efficient and economical Alkcell operation. These and other factors have been explored [14]. A third study has examined the potential for a continuous Alkcell Process and the feasibility of its operation locally, at or near the biomass growing site was established [15].

### BRIEF SUMMARY OF THE INVENTION

The Alkcell Process provides a method for the conversion of biomass (cellulosic material) such as agricultural waste like corn stover into glucose and its related cellulosic hydrolysis products. The process uses an alkali pretreatment of the biomass such as with sodium hydroxide (NaOH) in water solution. The pretreated biomass is then prepared for enzyme hydrolysis with cellulases or equivalent cellulosic hydrolytic enzymes. Following enzyme treatment of the pretreated biomass there is release of glucose and related hydrolysis products using a volume expansion technique. Glucose and its related products can then be concentrated using any applicable technique such as membrane or thermal concentration. The Alkcell Process uses commonly available materials and methods readily implemented near the biomass growing site but could be used at other locations as well. The advantage of the Alkcell Process is implementation at or near the biomass growing site such that the biomass does not need to be transported distances to special cellulose processing facilities. Likewise, the product, primarily glucose in water solution, can be transported for direct fermentation to ethanol, such as for use as liquid fuel, without further processing as would be required for untreated cellulosic materials. Furthermore, the product can be fermented at existing grain fermenting facilities so that special cellulose handling refineries would not be required. The Alkcell Process can be configured to a manual or powered process that can be done in individual steps or by continuous process.

### DESCRIPTION OF THE DRAWING

A schematic of the Alkcell Process as a continuous process is shown in DRAWING. Each critical component is identified. Specifics of the chemicals that would be used are not given in the drawing since they are identified elsewhere in the SPECIFICATION. The operation of the apparatus is given in the DETAILED DESCRIPTION OF THE INVENTION.

### DETAILED DESCRIPTION OF THE INVENTION

The Alkcell Process converts biomass, that is, cellulosic materials into glucose that can be fermented to produce bioethanol, that is, ethanol for use as a liquid fuel such as used in transportation vehicles. The process is a novel implementation of a chemical pretreatment of the biomass followed by enzyme hydrolysis using cellulases to produce glucose. The chemical pretreatment uses alkali chemicals such as sodium hydroxide in water solution heated to around T=100° C., that is, the boiling point of the water solution. Heating can be done with any energy source such as with a high btu propane burner. Pretreatment is conducted for up to 6 hours and is followed with a wash to adjust the pH to that required for cellulase activity. Washing can be done in two steps with water then buffer or in one step with more concentrated buffer, as is presumed in this description and its diagram to follow. The pretreated and pH adjusted biomass solids are then placed in a cellulase bath at room temperature, that is, near 20° C. for up to 1 hour to allow adsorption of the enzymes. The biomass solids are then placed in a buffer solution heated to around 60° C. to allow cellulose hydrolysis to proceed and glucose to be released. This step takes up to three hours but can be extended. The dilute glucose solution is transferred to a concentration tank and heated to boiling. The steam produced is released back into the hydrolysis tank to maintain temperature near 60° C. As boiling proceeds the glucose solution is concentrated and drained into a collection tank.

The Alkcell Process can be operated manually or powered or both and with separate steps or in a continuous fashion. Configured as a continuous process is it schematized as shown in the DRAWING. Six tanks are used with the first and fifth of material such as metal that is capable of withstanding direct flame from below. The size of the tanks depends on the size of the operation, that is, the amount of biomass being processed. Biomass is loaded into carriers such as metal or fiberglass containers that are carried through the series of tanks by means of a moving belt or chain such as a bicycle chain. The number of carriers can be adjusted to accommodate the amount of biomass being processed. The speed of the chain is adjusted to allow the desired residence time for the carriers in each tank. In the diagram the chain is moved by rotation of sprockets that are activated by a mechanical or power mechanism that are not shown in the diagram. Likewise, not shown is a structure to hold the sprockets. This could be constructed with two by two perpendicular slats anchored to themselves and a floor platform. Biomass remaining in its carrier at the end of enzyme hydrolysis in tank 4 is carried back to tank 1 for reprocessing as new biomass is added.

The dilute glucose solution in tank 4 is siphoned into tank 5 and heated to boiling. Heating can be done with any energy source such as a high btu propane heater. The steam generated is directed back into tank 4 to maintain temperature around 60° C. As the glucose solution is concentrated it is siphoned off into a collection tank. Concentration could also be done with membrane technology and tank 4 heated separately. The concentrated glucose solution can then be transported to a fermentation facility. The concentrated glucose solution can be fermented directly without further processing.

The novelty of the Alkcell Process is the use of readily available materials and methods that can be implemented without technically sophisticated processes and thus is suitable for operation locally, that is, near the biomass growing site. This avoids the need and cost to transport unprocessed biomass distances to fermentation facilities outfitted to handle raw biomass. The Alkcell Process product is a glucose solution that can be used as is at existing grain fermenting facilities to produce ethanol that could be used as a liquid fuel.

### REFERENCES

1. Lynd L R, Laser M S, Bransby D, Dale B E, Davison B, Hamilton, R, Himmel M, Keller M, McMillan J D, Sheehan J, Wyman CE (2008) How biotech can transform biofuels. Nature Biotech 26: 169-172.

2. Blanch H W, Simmons B A, Klein-Marcuschamer D (2011) Biomass deconstruction to sugars. Biotech J 6: 1086-1102.

3. Brethauer S, Wyman C E (2010). Review: continuous hydrolysis and fermentation for cellulosic ethanol production. Bioresource Technol 101: 4862-4874.

4. Kadam K L, Chin C Y (2009) Continuous biomass fractionation process for producing ethanol and low-molecular-weight lignin. Environ Prog Sustainable Energy 28: 89-99.

5. Yu T H E, English B C, Larson J A, De La Torre Ugarte D, Fu J S, Richards S (2011) Evaluating the Impacts of Biomass Feedstock Transportation on Air Quality: A Tennessee Case Study (No. 127657). University of Tennessee, Department of Agricultural and Resource Economics.

6. Hague M, Epplin F M (2012) Cost to produce switchgrass and cost to produce conversion rates. Biomass Bioenerg 46: 517-530.

7. Brechbill S C, Tyner W E, Heleji K E (2011) The economics of biomass collection and transportation and its supply to Indiana cellulosic and electric utility facilities. Bioenerg Res 4: 141-152.

8. Larson J A, Yu T-H, English B C, Mooney D F (2010) Cost evaluation of alternative switchgrass producing, harvesting, storing, and transporting systems and their logistics in the Southeastern USA. Agr Finance Rev 70: 184-200.

9. Savarese J J (2013) Local biomass processing is practical for facilitating fermentation to bioethanol. J Tech Innov Renew Energy (Publication details pending).

10. Klein-Marcuschamer D, Oleskowicz-Popiel P, Simmons B A (2012) The challenge of enzyme cost in the production of lignocellulosic biofuels. Biotechnol Bioeng 109:1083-1087.

11. Kumar R, Wyman C E (2009) Access of cellulase to cellulose and lignin for poplar solids produced by leading pretreatment technologies. Biotechnol Prog 25: 807-819.

12. Sathitsuksanohy N, George A, Zhang Y-H P (2013) New lignocellulose pretreatments using cellulose solvents: a review. J Chem Technol Biotechnol 88: 169-180.

13. South C R, Hogsett D A, Lynd R (1993) Continuous fermentation of cellulosic biomass to ethanol. Appl Biochem Biotech 39-40: 587-600.

14. Savarese J J (2013) Optimizing alkali-cellulase processing of biomass to glucose. Bioresources (Publication details pending).

15. Savarese J J (2013) Continuous alkali-cellulase processing of corn stover to glucose for bioethanol. Bioenerg Res (Publication details pending).

1. Process (Alkcell Process or alkali-cellulase process) to convert biomass (cellulosic material) to glucose using an alkali pretreatment followed by cellulase hydrolysis,
said process using materials and methods amenable to implementation at or near the biomass growing or storage site,
such that said process can be done in individual steps or by continuous process that uses mild temperatures not exceeding boiling of the water solutions employed in said process,
such that said temperatures can be achieved with thermal heating as with a gas fuel device such as a propane tank burner.

2. A process according to claim 1 that uses materials readily available commercially that present limited hazard to operators of said process, and
include dilute alkali water solutions such as aqueous sodium hydroxide, water based pH buffers such as citrate/sodium citrate, and cellulases in water solution.

3. A process according to claim 2 that does not require extensive technical training of operators.

4. A process according to claim 3 in which cellulase is allowed to adsorb onto the pretreated biomass in a small volume reactor, and
then said cellulase treated biomass is transferred to a volume expansion vessel containing pH buffer at a larger volume such as but not limited to a volume ten times that of the cellulose adsorption vessel,
such that there is the release of glucose.

5. The glucose water solution produced according to claim 4 is then concentrated such as by thermal heating, and
the steam produced then used to heat vessels of other stages of the said process, and
other concentration methods such as with membranes can also be employed.

6. The concentrated glucose solution produced according to claim 5 is then transported to grain fermenting facilities and used directly to produce ethanol that can be used as a liquid fuel.

7. The remaining solids from claim 6 are the residual solids that can be recycled t produce additional glucose directly from the residual solids or from a combination of fresh biomass and residual solids.
